(19) United States
(12) Patent Application Publication
(10) Pub. No.: US 2019/0358105 A1
(43) Pub. Date: Nov. 28, 2019

(54) SUPPORTING DEVICE FOR ENEMA ADMINISTRATION

(71) Applicant: Olga Song, Waterloo (CA)

(72) Inventor: Olga Song, Waterloo (CA)

(21) Appl. No.: 16/214,303

(22) Filed: Dec. 10, 2018

Related U.S. Application Data
(60) Provisional application No. 62/677,079, filed on May 28, 2018.

(30) Foreign Application Priority Data
May 28, 2018 (RU) 2018119546

(51) Int. Cl. A61G 7/05; A47C 21/00; A61M 3/02

(52) U.S. Cl. CPC A61G 7/0503; A61G 7/065; A61M 3/0225; A47C 21/00

(57) ABSTRACT

A supporting device for enema administration utility model may be used for enema administration in colon irrigation and can be used for self-administration of hydrocolonotherapy and other similar procedures. The device may be configured to retain or support a user in Fowler's position during enema administration. In some embodiments, the device may include a base having a supporting plate, and a scallop may be disposed on the supporting plate. A scallop may be configured with an arc shape which may generally resemble the arc shape of the bottom of a bowl of a toilet so that the scallop may be positioned around portions of the bowl to minimize spaces or gaps between the supporting plate and the bowl. One or more, such as four, legs may be coupled to the base. A backrest may be coupled to the base and the backrest may be angled relative to the supporting plate.

### CROSS REFERENCE TO RELATED APPLICATIONS

This application claims priority to and the benefit of the filing date of U.S. Provisional Application No. 62/677,079, filed on May 28, 2018, entitled “SUPPORTING ENEMA DEVICE”, and Russian Federation Application No. 2018119546, filed on May 28, 2018, entitled “Maintenance device for enema”, both of which are hereby incorporated by reference in their entirety.

### FIELD OF THE INVENTION

This patent specification relates to the field of sanitary-hygienic ware. More specifically, this patent specification relates to supporting devices for enema administration for colonic irrigation that can be used in self-administration of hydrocolonotherapy and other similar domiciliary medical procedures.

### BACKGROUND

The administration of an enema in the home may be necessary to relieve constipation or for colonic depletion for hygienic convenience. For self-administration of enema procedures, the user generally kneels or lays on their back side. To relax to properly irrigate the colon in these positions is fairly difficult. However, a disadvantage of the home administered enema is spillage of clysmic solution or feces making necessary cleaning and sanitizing the floor after each use.

Thus, to make the enema administration convenient and hygienic it is expedient to use devices supporting the user in semirecumbent position (Fowler's position).

Most devices commercially available for colonic irrigation are designed for professional application by certified hydriatricians. However, there exist supporting devices for domestic use. Specifically, U.S. design patents USD282781S, filed Feb. 25, 1986; USD367705, filed Mar. 5, 1996; and USD264881, filed Jun. 8, 1982, provide analogous designs of supporting devices in the form of a plastic board with a hole to remove wastes. In use, one end of the device rests on the lavatory bowl, and the other end—on a chair. In this structural configuration, the chair does provide secure and stable support of the user in inclined position; additionally, the wastes are removed directly along the surface of the device—this requires sanitation (cleaning and disinfection) of the board after each use. Besides, the structural design is not sufficiently flexible for use in different size lavatories because the board is too long to fit a small lavatory space.

U.S. Pat. No. 4,628,554, filed Dec. 16, 1986, provides a device with a similar structure, but the board is equipped with support feet. The device features similar drawbacks to the above devices. US patent application US20060247604 A1, filed Nov. 2, 2016, discloses a supporting device having a chair with inclined backrest to fit a toilet bowl. The chair has a hole to drain the wastes. A disadvantage of this device is that it also requires sanitation after each use.

The device of international application WO2007005539, filed Jan. 11, 2007, features a flat base with support leg and inclined backrest. One edge of the base is supported by a leg, the other rests on the bowl. The base also has a hole to remove the wastes. Like the other analogs discussed above, this device is not sufficiently hygienic in usage, as after each usage it requires sanitation. Furthermore, the device may result in injury of the user because of its poor stability characteristics.

Therefore, a need exists for novel supporting devices for enema administration for colonic irrigation that can be used in self-administration of hydrocolonotherapy and other similar domiciliary medical procedures.

### BRIEF SUMMARY OF THE INVENTION

A supporting device for enema administration is provided. The device may be configured to retain or support a user in Fowler's position during enema administration. In some embodiments, the device may include a base having a supporting plate, and a scallop may be disposed on the supporting plate. A scallop may be configured with an arc shape which may generally resemble the arc shape of the bottom of a bowl of a toilet so that the scallop may be positioned around portions of the bowl to minimize spaces or gaps between the supporting plate and the bowl. One or more, such as four, legs may be coupled to the base. A backrest may be coupled to the base and the backrest may be angled relative to the supporting plate.

In further embodiments, the backrest may be movably coupled to the base and may be moved into one or more angular positions relative to the supporting plate.

In still further embodiments, one or more legs may be movably coupled to the base so that the legs may be moved or folded into a compact state.

In yet further embodiments, the device may include one or more retainers which may be configured to prevent the device from being moved relative to, such as away from, a toilet. Optionally, a retainer may be configured to secure the device to portions of a toilet and/or to a structure, such as a wall, floor, furniture, etc., proximate to a toilet.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a top perspective view of an example of a supporting device for enema administration according to various embodiments described herein.

FIG. 2 illustrates a bottom perspective view of an example of a supporting device for enema administration according to various embodiments described herein.

FIG. 3 shows a bottom perspective view of an example of a supporting device for enema administration having legs moved or folded into a compact state according to various embodiments described herein.

FIG. 4 depicts a top perspective view of an example of a supporting device for enema administration with an exemplary toilet according to various embodiments described herein.

FIG. 5 illustrates a top reverse perspective view of an example of a supporting device for enema administration with an exemplary toilet according to various embodiments described herein.

FIG. 6 shows a top perspective view of another example of a supporting device for enema administration with an exemplary toilet according to various embodiments described herein.

FIG. 7 depicts a top reverse perspective view of another example of a supporting device for enema administration with an exemplary toilet according to various embodiments described herein.

FIG. 8 illustrates a top perspective view of still another example of a supporting device for enema administration with an exemplary toilet according to various embodiments described herein.

FIG. 9 shows a top reverse perspective view of still another example of a supporting device for enema administration with an exemplary toilet according to various embodiments described herein.

FIG. 10 depicts a top perspective view of an example of a supporting device for enema administration with an exemplary toilet in use by a user according to various embodiments described herein.

FIG. 11 illustrates a side elevation view of an example of a supporting device for enema administration with a backrest in a first angular position according to various embodiments described herein.

FIG. 12 shows a side elevation view of an example of a supporting device for enema administration with a backrest in a second angular position according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

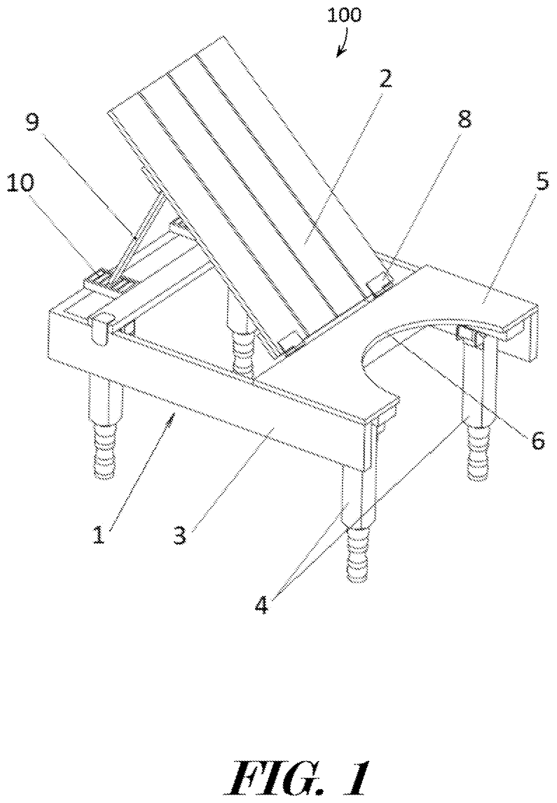

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new supporting device for enema administration is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

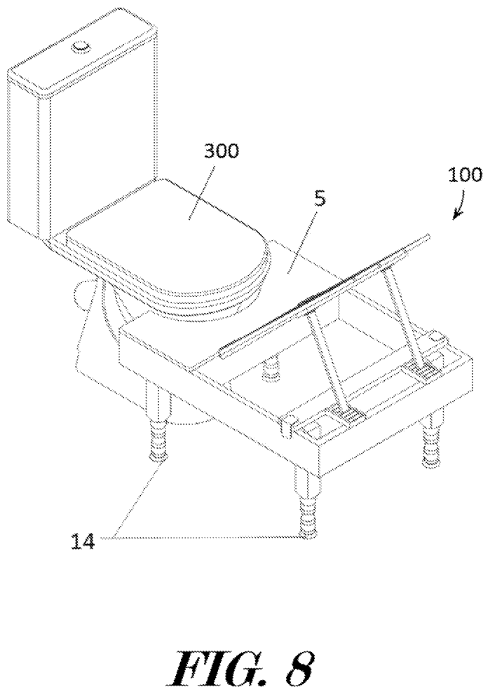

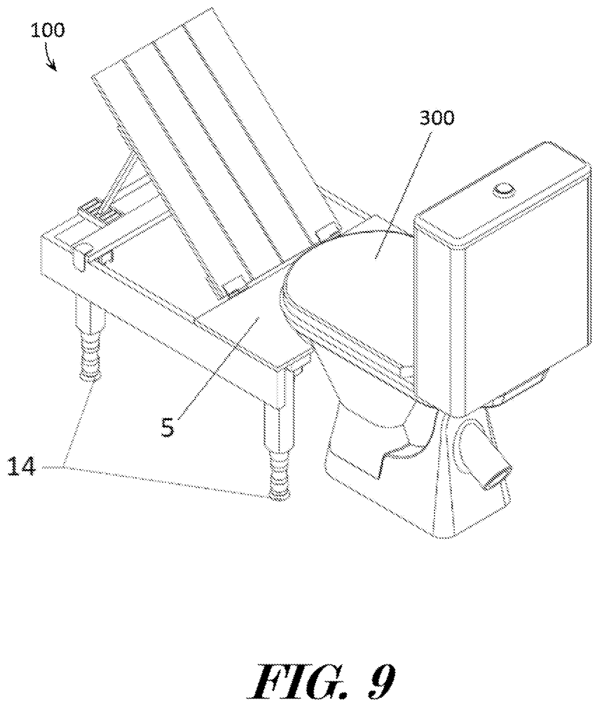

The present invention will now be described by example and through referencing the appended figures representing preferred and alternative embodiments. FIGS. 1-10 illustrate examples of a supporting device for enema administration (“the device”) 100 according to various embodiments. The device 100 may be configured to retain or support a user 200 in Fowler's position during enema administration.

In some embodiments, the device 100 may comprise a base 1 having a supporting plate 5, and a scallop 6 may be disposed on the supporting plate 5. One or more, such as four, legs 4 may be coupled to the base 1, and a backrest 2 may be coupled to the base 1.

In some embodiments, the base 1 may comprise one or more frame supports 3 which may be coupled together to form all or portions of the base 1 structure. The frame supports 3 may be configured in any shape or size to form all or portions of a base 1 in any shape or size. Preferably, the frame supports 3 may be made from or may comprise durable materials, such as wood, plastic, metal, etc., although any other material may be used.

The bae 1 may comprise a supporting plate 5 which may be coupled to one or more frame supports 3. A scallop 6 may be disposed on or in the supporting plate 5. Generally, a scallop 6 may comprise a recess that may be formed by portions of the supporting plate 5 to allow the device 100 to fit tight against a bowl 301 of a toilet 300 so that the outstanding rounded part of the bowl 301 engages with the scallop 6 to preferably form single supporting surface to accommodate the user. In preferred embodiments, a scallop 6 may be configured with an arc shape which may generally resemble the arc shape of the bottom of a bowl 301 of a toilet 300 so that the scallop 6 may be positioned around portions of the bowl 301 to minimize spaces or gaps between the supporting plate 5 and the bowl 301. In other embodiments, a scallop 6 may be configured with any other shape which may preferably allow the supporting plate 5 to conform to the shape of the bottom of a bowl 301 of a toilet 300 so that portions of the supporting plate 5 may be positioned proximate or in contact with portions of the bowl 301.

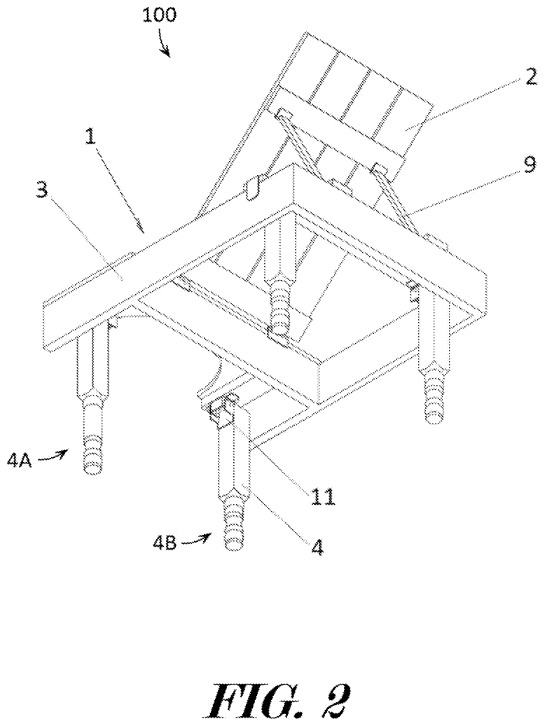

The device 100 may comprise one or more legs 4 which may be coupled to the base 1 and which may support the device 100 above a ground surface. In preferred embodiments, the device 100 may comprise four legs 4. A leg 4 may be configured in any shape and size. In some embodiments, a leg 4 may comprise a fixed length, while in preferred embodiments; a leg 4 may comprise an adjustable length so that the leg 4 may be moved into and between an extended position 4A (relatively longer length) and a retracted position 4B (relatively shorter length) as shown in FIG. 2. For example, a leg 4 may be extendable into and between a length of eight inches and a length of twenty four inches.

In some embodiments, an adjustable length leg 4 may comprise one or more leg sections which may be retracted into and extended out of one or more other leg sections in a telescoping manner. In other embodiments, an adjustable length leg 4 may comprise one or more leg sections which may be removably coupled together to form a leg 4 having various lengths. For example, by coupling three leg sections together a relatively shorter length leg 4 may be formed than a leg 4 having four or more leg sections.

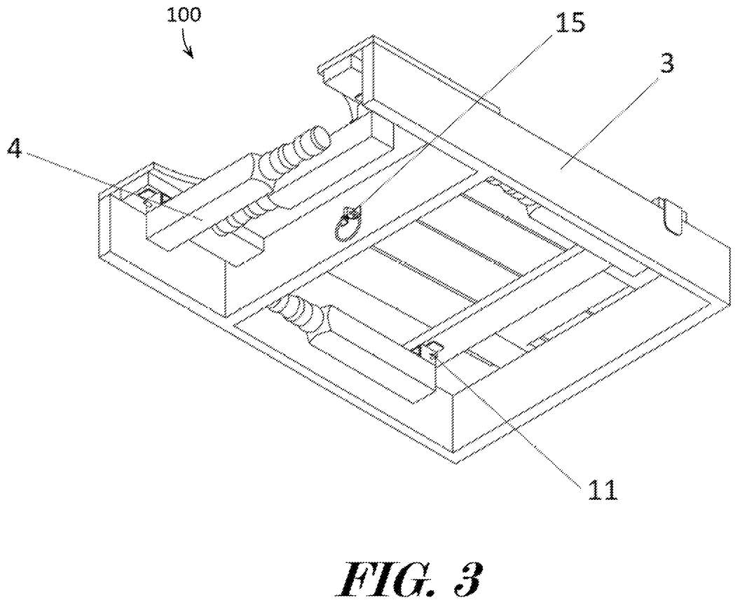

In further preferred embodiments, one or more legs 4 may be movably coupled to the base 1 so that the legs 4 may be moved or folded into a compact state, preferably making it possible to fold legs 4 inside the base 1 as shown in FIG. 3. In some embodiments, a leg 4 may be movably coupled to the base 1 via a leg hinge 11. A leg hinge 11 may comprise a piano hinge, butt hinge, barrel hinge, butt/Mortise hinge, case hinge, flag hinge, strap hinge, H hinge, HL hinge, piano hinge, butterfly hinge, flush hinge, concealed hinge, continuous hinge, T-hinge, double-acting hinge, Soss hinge, counterflap hinge, flush hinge, coach hinge, rising butt hinge, double action spring hinge, tee hinge, friction hinge, security hinge, cranked hinge or stormproof hinge, lift-off hinge, self closing or self positioning hinge, flexible material hinge, living hinge, or any other type or style of hinge or movable coupling suitable for movably joining a leg 4 to the base 1. In alternative embodiments, a leg 4 may be removably coupled to the base 1 so that the leg 4 and base 1 may be separated and re-attached from and to each other.

In some embodiments, a leg 4 may comprise a slip resistant material, such as rubber, silicone, etc., which may be positioned to contact a floor surface to prevent the leg 4, and therefore the device 100, from inadvertently moving relative to a toilet 300. In preferred embodiments, one or more legs 4 may each comprise a suction cup 14 which may be positioned to contact a floor surface to prevent the leg 4, and therefore the device 100, from inadvertently moving relative to a toilet 300. Generally, a suction cup 14 may comprise a device or object that uses the negative fluid pressure of air or water to adhere to nonporous surfaces, creating a partial vacuum. In alternative embodiments, a leg 4 and/or the base 1 may be coupled directly to a floor surface to prevent the device 100 from inadvertently moving relative to a toilet 300.

The device 100 may comprise a backrest 2 which may be configured to support the back of a user 200. Generally, a backrest 2 may be coupled to the base 1, optionally by being coupled to the supporting plate 5, so that the backrest 2 may be in an angular position relative to the supporting plate 5. In some embodiments, the backrest 2 may be movably coupled to the base 1 so that the backrest 2 may be moved into and between two or more angular positions relative to the supporting plate 5. In preferred embodiments, the backrest 2 may be movably coupled to the base 1 via a back hinge 8. Similar to a leg hinge 11, a back hinge 8 may comprise a hinge, such as a piano hinge, butt hinge, barrel hinge, butt/Mortise hinge, case hinge, flag hinge, strap hinge, H hinge, HL hinge, piano hinge, butterfly hinge, flush hinge, concealed hinge, continuous hinge, T-hinge, double-acting hinge, Soss hinge, counterflap hinge, flush hinge, coach hinge, rising butt hinge, double action spring hinge, tee hinge, friction hinge, security hinge, cranked hinge or stormproof hinge, lift-off hinge, self closing or self positioning hinge, flexible material hinge, living hinge, or any other type or style of hinge or movable coupling suitable for movably joining the backrest 2 to the base 1. In alternative embodiments, a backrest 2 may be removably coupled to the base 1 so that the backrest 2 and base 1 may be separated and re-attached from and to each other.

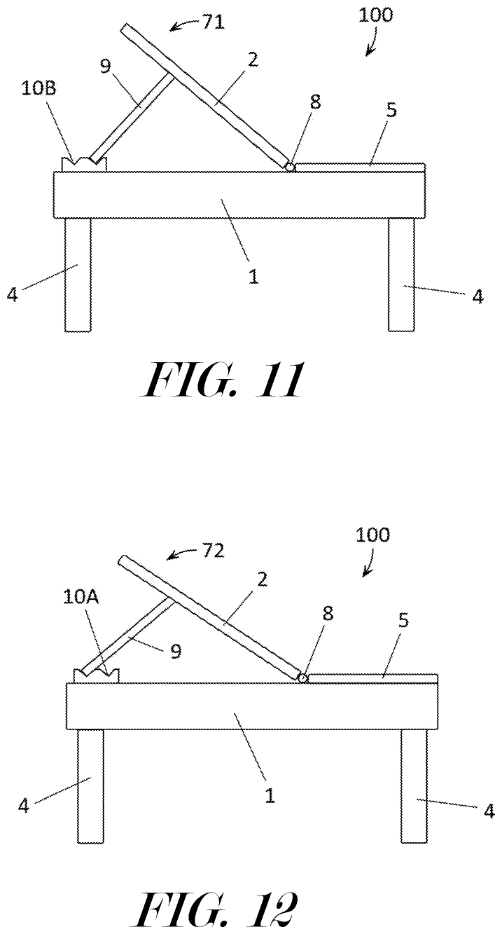

In some embodiments, the device 100 may comprise a brace 9 which may be configured to support the backrest 2 in an angular position 71, 72, relative to the supporting plate 5. Generally, a brace 9 may be configured to extend between the backrest 2 and the base 1 so that the brace 9 may prevent the backrest 2 from being moved towards the base 1. In preferred embodiments, the device 100 may comprise a one or more backrest locks 10, such as a first backrest lock 10A and a second backrest lock 10B, which may each be configured to receive or be coupled to a portion of a brace 9. Preferably, each backrest lock 10 may be positioned a different distance from the supporting plate 5 so that by positioning or coupling the brace 9 in different backrest locks 10, the brace 9 may support the backrest 2 in different angular positions 71, 72, relative to the supporting plate 5. For example and as shown in FIGS. 11 and 12, by positioning or coupling the brace 9 in a first backrest lock 10A that is relatively closer to the supporting plate 5, the brace 9 may support the backrest 2 in a first angular position 71 (FIG. 11) relative to the supporting plate 5 that is more angled than a second angular position 72 (FIG. 12) achieved by placing the brace 9 in a second backrest lock 10A that is relatively farther from the supporting plate 5.

In some embodiments, a backrest lock 10 may comprise a depression, aperture, channel, etc., into which portions of a brace 9 may be received. In other embodiments, a backrest lock 10 may comprise a fastener, such as a threaded fastener, pin, push-to-lock connection method, or any other fastener or connection method which may be used to secure or couple portions of a brace 9 to the base 1 or other element of the device 100.

In some embodiments, the device 100 may comprise one or more retainers 16 which may be configured to prevent the device 100 from being moved relative to, such as away from, a toilet 300. Optionally, a retainer 16 may be configured to secure the device 100 to portions of a toilet 300 and/or to a structure, such as a wall, floor, furniture, etc., proximate to a toilet 300.

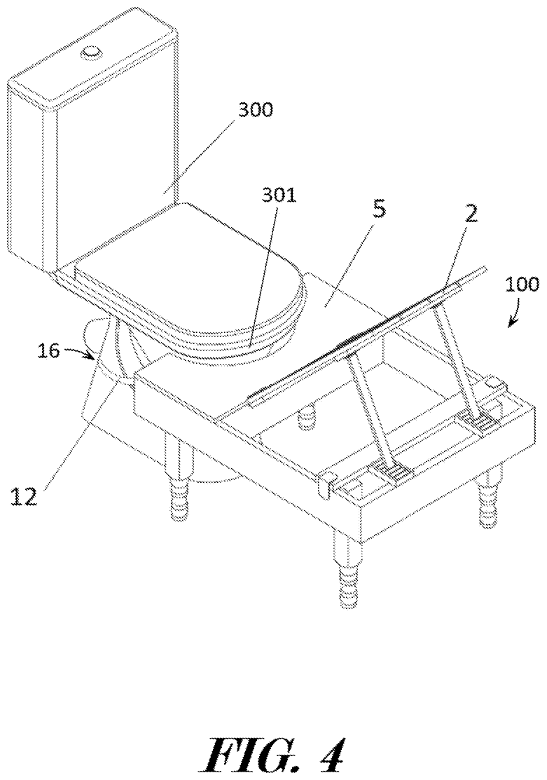

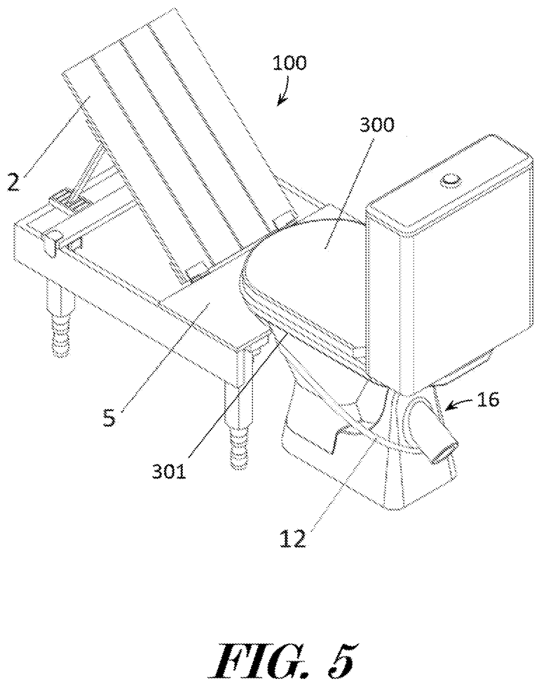

In some embodiments and as perhaps best shown by FIGS. 4 and 5, a retainer 16 may comprise a belt 12 made from one or more lengths of flexible material which may be configured to encircle or wrap around portions of a toilet 300 to couple the device 100 to the toilet 300. A belt 12 may be coupled to an element of the device 100, such as to a frame support 3 of a base 1, via a belt fastener 15. In some embodiments, a belt fastener 15 may comprise an attachment ring through which portions of a belt 12 may be inserted or otherwise coupled to. In other embodiments, a belt fastener 15 may comprise a hole or aperture in an element of the device 100, such a frame support 3 of a base 1, through which portions of a belt 12 may be inserted or otherwise coupled to. In alternative embodiments, a belt fastener 15 may comprise a threaded fastener, a rivet, heat bonding, adhesive, a turn-to-lock connection method, or any other fastener or connection method which may be used to couple a belt 12 to the base 1 or other element of the device 100.

Preferably, a belt 12 may be adjustable in length. A belt 12 may be made from or may comprise a flexible material, such as polypropylene webbing, nylon webbing, polyester webbing, various types of flexible plastics such as polyvinyl chloride, natural rubber, neoprene foam rubber, synthetic rubber material such as latex rubber, forms of the organic compound isoprene, Polyacrylate Rubber, Ethylene-acrylate Rubber, Polyester Urethane, a flexible plastic such as high-density polyethylene (HDPE), polyvinyl chloride (PVC), polypropylene (PP), Polystyrene (PS), Polycarbonate (PC), low density polyethylene (LDPE), synthetic fabrics such as polyester, nylon, rayon, acetate, spandex, spandex blends, and Kevlar, and natural fabrics such as coir, cotton, hemp, jute, canvas, flax, leather, linen, ramie, wool, silk, natural or synthetic rope, chain, or any other flexible material. In alternative embodiments, a belt 12 may comprise a substantially rigid material, such as aluminum, other metal and metal alloys, wood or other plant based materials, rigid plastics, or any other material or combinations of materials which may be substantially rigid so as to resist deformation.

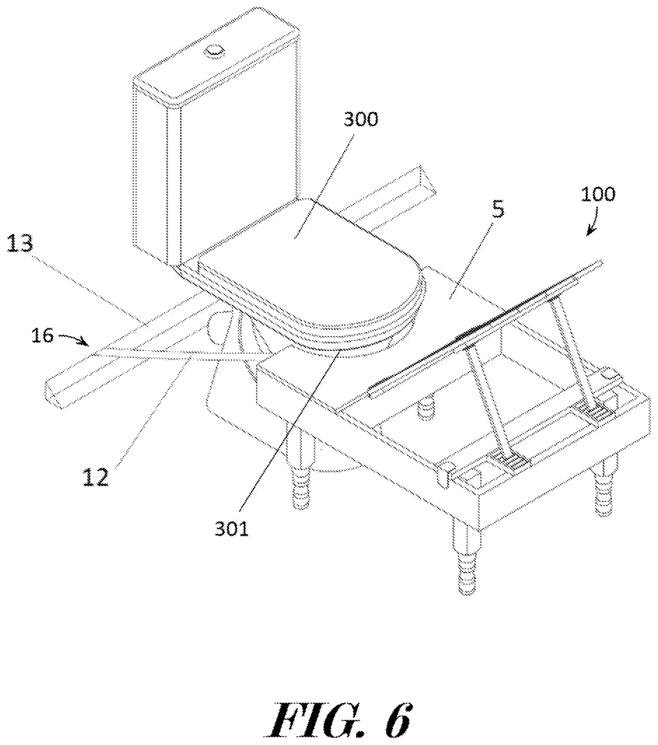

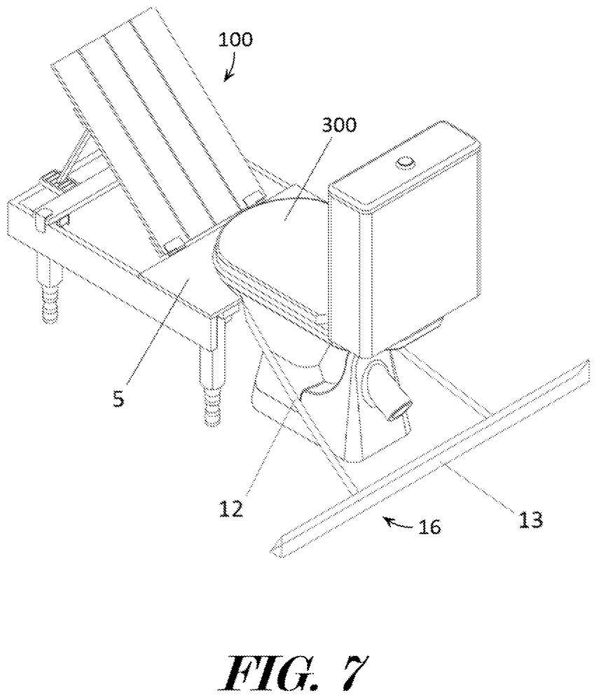

In further embodiments and as best shown in FIGS. 6 and 7, a retainer 16 may comprise a skirting plate 13 which may comprise a substantially rigid material which may be coupled to a structure, such as a wall, floor, furniture, etc., proximate to a toilet 300, and the belt 12 may then be coupled to the skirting plate 13 to prevent or resist the device 100 being moved relative to the toilet 300. In some embodiments, a skirting plate 13 may comprise a length of substantially rigid material that may extend past opposite sides of the toilet 300. For example, a skirting plate may comprise a three foot length of aluminum bar stock having one or more holes or apertures through which portions of a belt 12 may be coupled. In other embodiments, the retainer 16 may comprise two or more skirting plates 13 which may be separated from each other and to which portions of a belt 12 may be coupled. In alternative embodiments, a skirting plate 13 may comprise a fastener, such as a drywall anchor, screw or bolt, or any other fastener or coupling method which may be configured to couple a belt 12 to a toilet 300 and/or structure proximate to a toilet 300. For example, a skirting plate 13 may be configured as a suction cup which may be adhered to the toilet 300.

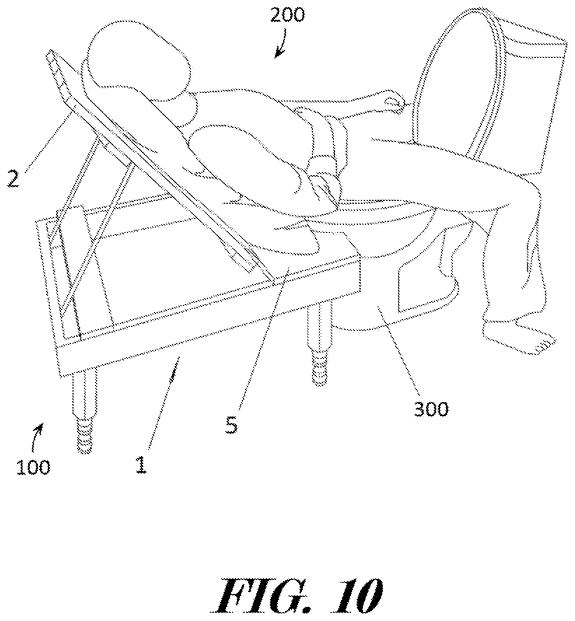

As perhaps best shown in FIG. 10, the device 100 may be configured to retain or support a user 200 in Fowler's position during enema administration. In some embodiments, the base 1 of the device 100 may be set tight against the bowl 301 of a toilet 300. Preferably, the bowl 301, supporting plate 5, and the backrest 2 may form a single surface to accommodate the user 200. The base 1 may be secured in this position by a suction cup 14 and/or a retainer 16.

The user 200 may sit on the device 100 and bowl 301, facing it, with feet on the floor and their back on the tilting backrest 2. Preferably, by positioning a brace 9, the user 200 can choose a desired angular position 71, 72, (angle of the tilt) convenient for him. In this position the user 200 can easily insert and remove the syringe cannula. During the procedure all wastes are deposited into the bowl 301 without contacting any of the elements of the device 100.

In this manner, the device 100 provides convenient position for the user 200 during the procedure in which waste may be evacuated directly into the bowl 301 of a toilet 300 without spillage risk.

Because of its convenience and inexpensiveness of the device 100, the domiciliary administration of enema can be an alternative to the colon therapy performed in medical establishments.

While some exemplary shapes and sizes have been provided for elements of the device 100, it should be understood to one of ordinary skill in the art that a base 1, backrest 2, frame support 3, leg 4, brace 9, backrest lock 10, belt 12, skirting plate 13, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A supporting device for enema administration, the device comprising:
a base comprising a supporting plate;
a scallop disposed on the supporting plate;
at least one leg coupled to the base; and
a backrest coupled to the base.

2. The device of claim 1, further comprising a retainer.

3. The device of claim 2, wherein the retainer comprises a belt configured to encircle a portion of a toilet.

4. The device of claim 2, wherein the retainer comprises a skirting plate, and wherein the belt is coupled to the skirting plate.

5. The device of claim 1, further comprising a suction cup that is coupled to the at least on leg.

6. The device of claim 1, wherein a leg is movably coupled to the base.

7. The device of claim 1, wherein a leg is adjustable in length.

8. The device of claim 1, wherein backrest is movably coupled to the base.

9. The device of claim 1, wherein backrest is movably coupled to the base via a hinge.

10. The device of claim 1, further comprising a brace configured to support the backrest in an angular position relative to the supporting plate.

11. The device of claim 10, further comprising a first backrest lock and a second backrest lock, wherein a portion of the brace is received in the first backrest lock to support the backrest in a different angular position than when the portion of the brace is received in the second backrest lock.

12. A supporting device for enema administration, the device comprising:
a base comprising a supporting plate;
a scallop disposed on the supporting plate;
at least one leg movably coupled to the base;
a backrest coupled to the base; and
a retainer.

13. The device of claim 12, wherein the retainer comprises a belt configured to encircle a portion of a toilet.

14. The device of claim 12, wherein the retainer comprises a skirting plate, and wherein the belt is coupled to the skirting plate.

15. The device of claim 12, further comprising a suction cup that is coupled to the at least on leg.

16. The device of claim 12, wherein a leg is adjustable in length.

17. The device of claim 12, wherein backrest is movably coupled to the base.

18. The device of claim 12, wherein backrest is movably coupled to the base via a hinge.

19. The device of claim 12, further comprising a brace configured to support the backrest in an angular position relative to the supporting plate.

20. The device of claim 19, further comprising a first backrest lock and a second backrest lock, wherein a portion of the brace is received in the first backrest lock to support the backrest in a different angular position than when the portion of the brace is received in the second backrest lock.
